(19) United States
(12) Patent Application Publication
(10) Pub. No.: US 2005/0261493 A1
(43) Pub. Date: Nov. 24, 2005

(54) Methods for the isolation and purification of ansamitocins

(76) Inventors: Mark Fulston, Redhill (GB); Anna L. Stefanska, King of Prussia, PA (US); Jan E. Thirkettle, Worthing (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/523,710

(22) PCT Filed: Aug. 7, 2003
(86) PCT No.: PCT/US03/24642

§ 371 Date: Feb. 4, 2005

Related U.S. Application Data
(60) Provisional application No. 60/401,877, filed on Aug. 8, 2002.

(51) Int. Cl. C07D 491/02

(52) U.S. Cl. 540/462

(57) ABSTRACT

This invention relates to processes for the preparation of ansamitocins, in particular ansamitocins that can be converted to maytansinol.

### FIELD OF THE INVENTION

This invention relates to processes for the preparation of ansamitocins, in particular ansamitocins that can be converted to maytansinol.

### BACKGROUND OF THE INVENTION

Highly cytotoxic maytansinoid drugs and their therapeutic use have been described in U.S. Pat. No. 5,208,020. These drugs can be prepared from ansamitocin precursors produced by fermentation of microorganisms such as Actinosynnema.

Processes for ansamitocin P-3 production from Actinosynemma spp. have been described in U.S. Pat. Nos. 4,162,940; 4,228,239; 4,356,265; and 4,450,234. In general, these methods require adding a filter aid and a water-miscible solvent to whole fermentation broth, removing solids and extracting the aqueous fraction with a water-immiscible solvent, concentrating and precipitating with petroleum ether, purifying the precipitate using silica chromatography, and crystallizing followed by further chromatography or re-crystallization. Patent application WO0177360 describes an improved method for ansamitocin production that utilizes fewer and more contained steps, namely extracting the fermentation broth with an aromatic hydrocarbon solvent, concentrating the extracted ansamitocins and purifying the ansamitocins by crystallization.

The concentration of the extracted ansamitocins is generally carried out using large plant such as falling film evaporators, which are difficult to contain, costly and in addition may cause thermal degradation of the product. There is a need to provide a simple, effective, contained means of concentration of the solvent extracted ansamitocins. Additionally, because of the extremely toxic nature of the ansamitocin compounds, safer, alternative purification procedures, utilizing simpler and more contained stages, with fewer manipulations are of benefit for large-scale production operations.

### SUMMARY OF THE INVENTION

Aspects of the present invention include improved methods of preparing purified ansamitocins.

A method for capture of ansamitocins onto silica gel achieving concentration and purification.

A method whereby no evaporative steps are required in the isolation process.

A method for purification of solvent extracted broth by column or batch treatment with activated carbon, using a solvent system that retains impurities but not ansamitocins.

A method for purification of toluene extract by column or batch treatment with activated carbon in a toluene/polar alcohol mixture.

A method of purification of eluate from silica chromatography by column or batch treatment with activated carbon, using a solvent system that retains impurities but not ansamitocins.

A method of purification of eluate from silica chromatography by column or batch treatment with activated carbon, in a toluene/polar alcohol mixture.

A method for crystallisation of ansamitocins using a halogenated hydrocarbon and a polar solvent

### DETAILED DESCRIPTION OF THE INVENTION

One embodiment of the method of the invention is to achieve concentration of solvent extracted ansamitocins by capture onto silica gel. Conventionally silica chromatography is carried out by applying a small volume of concentrated feed material to a column, followed by chromatographic separation and elution. One aspect of the invention is to pass a large volume of dilute feed onto a silica column, retaining the ansamitocins on the silica. This is followed by elution in a small volume of solvent with concomitant purification and concentration.

All forms of silica column may be used but radial compression, cartridge based systems are preferred, due to their containment, speed and the high surface activity of the grade of silica. The column may be run in conventional (pressurised feed) mode, or preferably by applying a vacuum to the permeate and maintaining the feed at atmospheric pressure, thus reducing the risk of leakage. The silica is eluted with a toluene/methanol mixture concentration chosen to remove P-3 but retain unwanted material. This may be achieved using a solvent gradient such as within the range 1-15% methanol (preferably 2-6%), or isocratically with preferably 3-5% methanol, most preferably 4%.

The silica capture method may be further refined to avoid the need for any evaporative steps whatsoever in the procedure, which allows the use of simple, easy to contain plant. This is achieved by directly crystallising the bulked eluate fractions from the column by the addition of heptane or a similar low polarity solvent as exemplified in Example 1.

Alternatively, the concentrated ansamitocin eluate from the silica capture stage may either be evaporated to dryness ready for crystallisation using methods exemplified herein or as described in previous patent applications. The silica eluate may first be treated with activated carbon, (such as SK1 from CPL), [Stirling House, 2 Park St, Wigan, Lancs WN3 5HE.UK], either by passage through a column or by batch treatment. In the method of this invention, carbon does not retain the ansamitocins, which pass through, leaving impurities adsorbed to the carbon.

Although the modes of purification are different for silica and carbon and different impurities are removed, these two adsorption methods produce equivalent quality product after crystallisation. Due to the difference in selectivity between silica and activated carbon, these steps may be advantageously combined as described in Examples 2 and 3.

The toluene extract is ideally partially concentrated (approximately 10-fold), prior to carbon treatment, but the step may be carried out at any degree of concentration. This method involves the addition of modifier solvents (e.g. a polar alcohol, ideally methanol), to the toluene, to optimal concentrations (typically 2-8%, ideally 4%). The concentration is chosen to prevent adsorption of the ansamitocins onto the carbon whilst maximising the retention and removal of impurities.

Treatment with activated carbon may also be used as an alternative or additional purification step when using conventional evaporative concentration of toluene extract, as described in Example 5. This is a fast, simple procedure whereby the concentrated feed is made up to 4% methanol/toluene prior to loading onto a pre washed SK1 carbon column and percolated through the carbon. Impurities are adsorbed from the crude feed, and the ansamitocin containing percolate is collected as one fraction. This step may also be carried out by batch treatment with activated carbon.

Crystallization is used to purify the ansamitocins and preferentially reduce levels of unwanted ansamitocins. The crystallization may be performed using the methods described in previous ansamitocin patent applications, or it may be carried out using a halogenated hydrocarbon, preferably dichloromethane (DCM) as described in Example 4. This step may be used to purify crude extracts, carbon treated extracts, silica treated extracts or recrystallise impure crystals produced from other solvent systems. Due to the high solubility of ansamitocins in DCM, small volumes of solvent may be used as opposed to the large volumes/low concentrations required for ethyl acetate based systems. The crystallization is carried out using a non-polar co-solvent such as heptane to control crystal growth and maximise yield by reducing the solubility in the solvent. The crystallisation is ideally carried out using a highly concentrated solution of ansamitocins in DCM at 35-45° C., in the range 50-200 mg/mL, preferably in the range 100-180 mg/mL P-3] and then cooling to ambient temperature. Alternatively, the crystallisation may be carried out with the addition of 1-3 vols of heptane, preferably 1.5-2 volumes. Cooling to 5-10° C. post heptane addition may be used to increase yield. These ratios of solvent maximise yield whilst avoiding co-precipitation of impurities. This system also achieves purification by selectively crystallising ansamitocin P-3 and other desirable maytansinol esters and lowering the levels of ring-modified unwanted ansamitocins.

In the examples described, the ansamitocins were produced by fermentation of Actinosynnema pretiosum ATCC 31565. The fermentation and toluene extractions were carried out essentially as described in the international application WO0177360.

Quantitation and qualification of ansamitocins was carried out by HPLC, using the methods described in application WO0177360.

### EXAMPLES

### Example 1

### Direct Capture of Ansamitocins in Crude Toluene Extract onto a Silica Cartridge, Evaporation and Crystallisation

A Biotage Flash 75S™ cartridge system (75 mm×15 cm), [Biotage UK Ltd, 15, Hartforde Court, John Tate Road, Foxholes Business Park, Herts SG13 7NW, UK], containing 200 g silica (KP-Sil™), (cartridge volume 250 mL), was set up so that it could be run in both pressure and/or vacuum mode. Toluene extract (˜25-26 L), of Actinosynnema pretiosum whole broth containing approximately 2.6 g ansamitocin P-3, was loaded onto the Biotage cartridge at 200 mL/min. The flow was controlled by adjusting the vacuum level to the permeate vessel. Cartridge percolate fractions were assayed after 5 and 20 L to check for ansamitocin P-3. There was no breakthrough of P-3.

After the toluene extract had been loaded, the cartridge system was switched to run in pressure mode (20 psi), and eluted with 4% methanol/toluene, at approx. 100 mL/min. The eluate was collected as 250 or 500 mL fractions.

Fractions containing ansamitocin P-3 (F6-9) were bulked to give 1500 mL of eluate. 2.67 g P-3 was recovered. The eluate was divided into 2×750 mL aliquots.

750 mL of the silica eluate was taken to dryness on a rotary evaporator in a round bottom flask. 2 mL of methanol was added to the residue, followed by 30 mL of ethyl acetate. The product was stoppered and transferred to a 50° C. heated water bath and stirred until all the material had dissolved. Pre-warmed heptane (approximately 25 mL, 50° C.), was slowly added, watching the solvent mix for initial signs of clouding, at which point the flask was removed from the water bath and allowed to cool to room temperature (20° C.). The flask was left to stand and crystals started to form. The flask was returned to the water bath and additional warm heptane added up to 64 mL (≡2×vol. of methanol+ethyl acetate). The flask was allowed to cool and left overnight. Large quantities of clear crystals were formed.

The mother liquor, containing ˜5% P-3, was decanted, and the crystals washed with 20 mL of fresh ethyl acetate/heptane 1:3, and the wash was also decanted. The crystals were dried by slowly rotating the flasks under vacuum at 40° C. on a rotary evaporator. 1.4 g of crystals were obtained, (1.22 g P-3). The crystals contained 5.7% P-2; 86.0% P-3; 7.9% P-4. [Overall P-3 yield approximately 92%.]

### Example 2

### Carbon Treatment of Silica Eluate, Evaporation and Crystallisation

750 mL of the silica eluate in 4% MeOH, prepared as described in Example 1, was loaded onto a 31 g SK1 carbon column (30×120 mm), which had been prepared and washed with 4% methanol/toluene. The percolate was collected in 10 mL fractions and the column finally washed with a further 30 mL of fresh 4% methanol/toluene. The percolate fractions were bulked and evaporated to dryness on a rotary evaporator in a round bottom flask.

2 mL of methanol was added to the residue, followed by 30 mL of ethyl acetate. The product was stoppered and transferred to a 50° C. heated water bath and stirred until all the material had dissolved. Pre-warmed heptane (approx. 25 mL, 50° C.), was slowly added, watching the solvent mix for initial signs of clouding at which point the flask was removed from the water bath and allowed to cool to room temperature (20° C.). The flask was left to stand and crystals started to form. The flask was returned to the water bath and further warm heptane added up to 64 mL′ (≡2×vol. of methanol+ethyl acetate). The flask was allowed to cool and left overnight. Large quantities of clear crystals were formed.

The mother liquor, containing ˜5% P-3, was decanted and the crystals washed with 20 mL of fresh ethyl acetate/heptane 1:3, and the wash was also decanted. The crystals were dried by slowly rotating the flasks under vacuum on a rotary evaporator at 40° C. 1.23 g of crystals were obtained, (1.07 g P-3). The crystals contained 5.7% P-2; 87.0% P-3; 7.3% P-4. [Overall P-3 yield approximately 80.0%.]

### Example 3

### Direct Capture of Ansamitocins in Crude Toluene Extract onto a Silica Cartridge, Carbon Treatment

Toluene extract (45 L), of whole broth of Actinosynnema pretiosum containing 168 mg/L ansamitocin P-3 (7.56 g), was loaded onto a Biotage Flash 75S™ cartridge system containing 200 g of silica (KP-Sil™), using pressure (20 psi), at ˜200 mL/min. A sample was withdrawn and assayed every 5 L from the percolate to check for signs of ansamitocin P-3 breakthrough.

### Example 5

### Evaporative Concentration of Crude Toluene Extract, followed by Carbon Treatment, Evaporation and Crystallisation

Toluene extract (12.5 L), of whole broth of Actinosynnema pretiosum containing 98 mg/L ansamitocin P-3 (1.23 g), was concentrated 10-fold on a rotary evaporator to 1.25 L. The concentrate was made up to 4% methanol/toluene prior to loading onto a pre washed SK1 carbon column (33×330 mm; 10-15 mL/min.). The percolate was dried on a rotary evaporator to give 4.8 g of material. This was taken up in 1.8 mL methanol+24 mL ethyl acetate and heated in a water bath to 50° C. Warm heptane (52 mL), was slowly added then the flask was allowed to cool to room temperature. Crystals formed in the flask. The flask was left at 40° C. for approximately 1 hr. HLPC analysis indicated that 5% P-3 remained in the mother liquor. The mother liquor was decanted and the crystals washed with 10 mL ethyl acetate/heptane 1:3. The wash was decanted and the crystals dried under vacuum on a rotary evaporator at 40° C. 1.06 g of crystals were obtained, containing 0.92 g P-3. The crystals contained 5.7% P-2; 86.8% P-3; 7.5% P-4. [Overall yield 86.2%.]

1. A method for capture of ansamitocins onto silica gel achieving concentration and purification.

2. A method whereby no evaporative steps are required in the isolation process.

3. A method for purification of solvent extracted broth by column or batch treatment with activated carbon, using a solvent system that retains impurities but not ansamitocins.

4. A method for purification of toluene extract by column or batch treatment with activated carbon in a toluene/polar alcohol mixture.

5. A method of purification of eluate from silica chromatography by column or batch treatment with activated carbon, using a solvent system that retains impurities but not ansamitocins.

6. A method of purification of eluate from silica chromatography by column or batch treatment with activated carbon, in a toluene/polar alcohol mixture.

7. A method for crystallisation of ansamitocins using a halogenated hydrocarbon and a polar solvent.
